(19) United States
(12) Patent Application Publication
(10) Pub. No.: US 2017/0215415 A1
(43) Pub. Date: Aug. 3, 2017

(54) OXIDIZING DISINFECTANT FORMULATION AND METHODS OF USE

(71) Applicant: David Reay, Lindon, UT (US)

(72) Inventor: David Reay, Lindon, UT (US)

(21) Appl. No.: 15/416,748

(22) Filed: Jan. 26, 2017

Related U.S. Application Data
(60) Provisional application No. 62/288,649, filed on Jan. 29, 2016.
(60) Provisional application No. 62/450,180, filed on Jan. 25, 2017.

(51) Int. Cl. A01N 33/12; A61L 2/18; A61L 2/22; C11D 3/30; C11D 3/48; A01N 25/16; C11D 3/20; C11D 1/66; C11D 3/33; A01N 25/08; A01N 25/02; A61L 2/23; C11D 3/39

(52) U.S. Cl. CPC A01N 33/12; A61L 2/23; A61L 2/18; A61L 2/22; C11D 3/30; C11D 3/48; C11D 3/3945; C11D 3/2086; C11D 1/66; C11D 3/33; A01N 25/08; A01N 25/02; A01N 25/16

(57) ABSTRACT

A composition that is useful for disinfecting surfaces, instruments, or equipment that may comprise an oxidizer precursor, a pH adjusting agent, an acetylating agent, a chelating agent, and a quaternary ammonium salt is described. The composition may be dispensed in a variety of ways, for example, such as a sprayed solution, a fog, or a foam.

### BACKGROUND

1. Related Applications

This application claims the benefit of: U.S. Provisional Patent Application Ser. No. 62/288,649, filed Jan. 29, 2016; and U.S. Provisional Patent Application Ser. No. 62/450,180, filed Jan. 25, 2017; which are hereby incorporated by reference in their entireties.

2. The Field of the Invention

This invention relates to disinfectants, and more particularly, oxidizing disinfectants suitable for cleaning and sterilizing a variety of surfaces, instruments and equipment, thereby aiding the control and treatment of human and animal diseases associated with contaminated surfaces, wounds, instruments and equipment.

3. Background

The current invention may have applications in the field of human and animal health. In one embodiment, an oxidizer (e.g., peracetic acid, hydrogen peroxide) and a quaternary ammonium compound may be utilized for the control and/or treatment of human and animal diseases associated with contaminated surfaces, wounds, instruments and equipment.

Peracetic acid (C₂H₄O₃) in an aqueous solution is a mixture that may be described as further comprising acetic acid (CH₃COOH) and hydrogen peroxide (H₂O₂). Typically, peracetic acid (hereinafter “PAA”) is produced by reacting acetic acid and hydrogen peroxide. It is also well known to generate a liquid solution comprising PAA starting from the dissolution of a powdered mixture (U.S. Pat. No. 7,291,276; UK patent application No. 2,355,198; FR Patent No. 2,728,171; Canadian Pat. No. 2,569,025, which are all incorporated herein by reference in their entireties as to all they teach).

Quaternary ammonium cations, also known as quats, are positively charged polyatomic ions of the structure NR₄⁺, R being an alkyl group or an aryl group. Unlike the ammonium ion (NH₄⁺) and the primary, secondary, or tertiary ammonium cations, the quaternary ammonium cations are permanently charged, independent of the pH of their solution. Quaternary ammonium salts or quaternary ammonium compounds (called quaternary amines in oilfield parlance) are salts of quaternary ammonium cations with an anion. Quaternary ammonium compounds have also been shown to have antimicrobial activity. Certain quaternary ammonium compounds, especially those containing long alkyl chains, are used as antimicrobials and disinfectants. For example and not by way of limitation, benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide. Also good against fungi, amoeba, and enveloped viruses, quats act by disrupting the cell membrane. Quaternary ammonium compounds are lethal to a wide variety of organisms except endospores, Mycobacterium tuberculosis and non-enveloped viruses.

In contrast to phenolics, quaternary ammonium compounds are not very effective in the presence of organic compounds. Yet, they are very effective in combination with phenols. Quaternary ammonium compounds are deactivated by soaps, other anionic detergents, and cotton fibers. Also, they are not recommended for use in hard water. Effective levels are at 200 ppm. They are effective at temperatures up to 212° F. (100° C.). Quaternary ammonium salts are popular chemicals used in foodservice industry as sanitizing agents.

What is needed is a stable, effective disinfectant that can be used in a variety of ways to help clean and disinfect surfaces, instruments and equipment in a variety of situations that may include agricultural buildings, veterinary hospitals and facilities, food processing plants, medical and dental establishments, and the like.

### BRIEF SUMMARY OF THE INVENTION

In accordance with the foregoing, certain embodiments of a composition or formulation may be used by various methods in accordance with the invention to provide a formula and system for disinfecting various surfaces, instruments and equipment.

In one embodiment, the stabilized composition or formulation may be a powdered product that generates peracetic acid in situ and release quaternary ammonium chloride in water. The stabilized composition may be formulated to disinfect medical instruments and medical surfaces. It may also disinfect surfaces in food, beverage and meat processing and packaging plants. The stabilized composition disinfects equipment and surfaces in animal farms. It may also be used as a cleaning and disinfecting agent to clean and disinfect the cages of various animals. It is an ideal surface disinfectant in veterinary, medical and dental establishments. It is also ideal for use as a topical disinfectant for animal hygiene and wound care. In one embodiment, it may be a foaming, wide-spectrum disinfectant, and used on a regular basis it can kill virus, bacteria, yeast and mold.

In one embodiment, a disinfectant may include a composition of water soluble ingredients including at least one oxidizer (e.g., peracetic acid and hydrogen peroxide) and at least one quaternary ammonium salt.

In one embodiment, a composition for use in various disinfecting applications comprises an oxidizer precursor in powdered form, a pH adjusting agent in powdered form, and an acetylating agent in powdered form, wherein the oxidizer precursor, the pH adjusting agent, and the acetylating agent are mixed together to produce an homogenous mixture.

In one embodiment, a composition may further comprise a chelating agent in powdered form, wherein the chelating agent is mixed into the homogenous mixture. In another embodiment, a composition may further comprise a quaternary ammonium salt in powdered form, wherein the quaternary ammonium salt is mixed into the homogenous mixture. In another embodiment, a composition may further comprise a surfactant included in the homogenous mixture.

In one embodiment, the homogenous mixture may be mixed with water to produce an oxidizing, disinfecting solution.

In one embodiment, an oxidizer precursor comprises about 30-80% w/w of the homogenous mixture, a pH adjusting agent comprises about 10-40% w/w of the homogenous mixture, and an acetylating agent comprises about 10-40% w/w of the homogenous mixture. In another embodiment, a chelating agent comprises about 0.01-10% w/w of the homogenous mixture. In another embodiment, a quaternary ammonium salt comprises about 1-10% of the homogenous mixture.

In one embodiment, an oxidizer precursor may be selected from the group consisting of a peracetic acid precursor, a hydrogen peroxide precursor, a persalt, or hydrogen peroxide. In another embodiment, a quaternary ammonium salt may be selected from the group consisting of benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, didecyl dimethyl ammonium bromide, or dioctyl dimethyl ammonium bromide.

In one embodiment, a composition that may be used in disinfecting applications may comprise sodium percarbonate, citric acid, ethylenediamenetetraacetic acid (EDTA), tetraacetylethylenediamine (TAED), alkyl dimethyl benzyl ammonium chloride, alkyl dimethyl ethylbenzyl ammonium chloride, and ethoxylated alcohol, with all components mixed together to form a homogenous mixture in the form of a powder. Such a composition may further include a surfactant. Such a composition may be dissolved in water to form a solution suitable for multiple disinfecting applications.

In one embodiment, a method for disinfecting surfaces, instruments, or equipment may comprise providing a homogenous mixture, wherein the homogenous mixture comprises an oxidizer precursor in powdered form, a pH adjusting agent in powdered form, and an acetylating agent in powdered form. Such a method may further include adding water to the homogenous mixture to produce a disinfecting solution, dispensing the disinfecting solution, and allowing the disinfecting solution to remain on the treatment surface as dispensed for at least five minutes.

In one embodiment, a method may utilize an homogenous mixture that further includes a chelating agent. In another embodiment, a method may utilize an homogenous mixture that further includes a surfactant.

In one embodiment, a method may dispense a resultant disinfecting solution as a fog, which fog may be produced by utilizing an electrostatic fogging machine. In another embodiment, a method may dispense a resultant disinfecting solution as a foam.

### BRIEF DESCRIPTION OF THE DRAWINGS

The foregoing features of the present invention will become more fully apparent from the following description and appended claims, taken in conjunction with the accompanying drawings and experimental data. Understanding that these drawings and data depict only typical embodiments of the invention and are, therefore, not to be considered limiting of its scope, the invention will be described with additional specificity and detail through use of the accompanying drawings and data in which:

FIG. 1 illustrates a method for use of a disinfectant as described herein.

### DETAILED DESCRIPTION OF THE PREFERRED EMBODIMENTS

It will be readily understood that the components and steps of the present invention, as generally described and illustrated herein, could be arranged and designed in a wide variety of different configurations. Thus, the following more detailed description of the embodiments of the system and method of the present invention, as may also be represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

In one embodiment of the invention, a composition or formulation of water soluble ingredients for the control of human and animal pathogens may comprise at least one oxidizer (e.g. peracetic and hydrogen peroxide) and at least one quaternary ammonium chloride.

In certain embodiments, a composition or formulation may include a quaternary that is: benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, and/or domiphen bromide. The quaternary may also be a newer quaternary ammonium compounds (i.e., fourth generation), referred to as twin-chain or dialkyl quaternaries (e.g. didecyl dimethyl ammonium bromide and dioctyl dimethyl ammonium bromide), which purportedly remain active in hard water and are tolerant of anionic residues. The bactericidal action of the quaternaries has been attributed to the inactivation of energy-producing enzymes, denaturation of essential cell proteins, and disruption of the cell.

In certain embodiments, a composition may include at least one oxidizer, such as peracetic acid or hydrogen peroxide (or a mixture thereof).

In another embodiment, a composition or formulation of water soluble ingredients for the control of human and animal pathogens on surfaces, equipment and instruments may comprise a peracetic acid precursor, such as, hydrogen peroxide or hydrogen peroxide precursor or a mixture thereof, and a pH adjusting agent, and an acetylating agent, and at least one at least one quaternary ammonium compound. Such a composition may generate peracetic acid (PAA) in situ and the quaternary ammonium compound may be dissolved upon addition of water. Both quaternary ammonium compounds and oxidizers are stable in solution and do not decompose each other.

In certain embodiments, a composition may include a quaternary ammonium compound that is: benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, and/or domiphen bromide. A quaternary ammonium compound may also be a “newer” quaternary ammonium compound (i.e., fourth generation), referred to as twin-chain or dialkyl quaternaries (e.g. didecyl dimethyl ammonium bromide and dioctyl dimethyl ammonium bromide), which purportedly remain active in hard water and are tolerant of anionic residues. A quaternary ammonium compound may be a first generation up to a fourth generation quaternary compound.

In certain embodiments, a composition may include a quaternary compound that is composed of n-Alkyl (60% C14, 30% C16, 5% C12, 5% C18) Dimethyl Benzyl Ammonium Chloride and/or n-Alkyl (68% C12, 32% C14) Dimethyl Ethylbenzyl Ammonium Chloride.

In certain embodiments, a composition may include a quaternary ammonium chloride composed of a dry compound, such as a quaternary ammonium dried upon the surface of urea granules.

In one embodiment, a composition of water soluble ingredients for the control of human and animal pathogens on surfaces, equipment and instruments may comprise a peracetic acid precursor, such as a solid hydrogen peroxide precursor, and a pH adjusting agent, and an acetylating agent, and a chelating agent, or a sequestering agent. Such a composition may also include at least one quaternary ammonium chloride. Such a composition may generate in situ peracetic acid (PAA) upon addition of water.

In certain embodiments, a powdered composition may include a sequestering agent (a chelating agent). Such a powdered composition may include a chelating agent such as an inorganic acid, an organic acid, or a mixture of at least two acids that are inorganic acids and/or organic acids. Such a powdered composition may include a chelating agent, such as ethylenediaminetetraacetic acid (EDTA), nitrilotriacetic Acid (NTA), diethylene triamine pentaacetic acid (DTPA), 1-hydroxyethane (1,1-diylbiphosphonic acid) (HEDP), diethylenetriaminepentakis (methylenephosphonic acid) (DTPMP), amino trimethylene phosphonic acid (ATMP), ethylene diamine tetra (methylene phosphonic acid) (EDTMPA Solid), phosphonobutane tricarboxylic acid (PBTCA), phosphonates, or mixtures thereof.

In certain embodiments, a powdered composition may include an acylating agent such as tetraacetyl glycoluril (TAGU), tetraacetylethylendiamine (TAED), diacetyldioxohexahydratriazine (DADHT), or mixtures thereof.

In certain embodiments, a powdered composition may include an acylating agent such as acetylsalicylic acid or tetraacetylethylenediamine (TAED).

In certain embodiments, a powdered composition may include an acetylating agent, such as an organic acid containing at least one acyl group which is susceptible to perhydrolysis. For example and not by way of limitation, an acetylating agent may be a N-acyl compound or a O-acyl compound containing an acyl radical R—CO—wherein R is an aliphatic group having from 5 to 18 carbon atoms, or an alkylaryl group having from 11 to 24 carbon atoms, with 5 to 18 carbon atoms in the alkyl chain, or R is an aliphatic group having from 5 to 12 carbon atoms.

In certain embodiments, a powdered composition may include a solid hydrogen peroxide precursor, such as a persalt. Such a persalt may be sodium perborate, sodium percarbonate, ammonium percarbonate, sodium peroxyhydrate, calcium peroxide, sodium peroxide, sodium perborate monohydrate, sodium perborate tetrahydrate, sodium persulfate, potassium monopersulfate, perphosphate, magnesium peroxide, zinc peroxide, urea hydrogen peroxide, perhydrate of urea, thiourea dioxide, or mixtures thereof.

In certain embodiments, a powdered composition may include a pH adjusting agent such as an organic acid or an inorganic acid. Such a pH adjusting agent may be citric acid.

In one embodiment, a composition of water soluble ingredients for the control of human and animal pathogens associated with contamination of surfaces, instruments and equipment may include a peracetic acid precursor, such as hydrogen peroxide, hydrogen peroxide precursor or a mixture thereof, and a pH adjusting agent, and an acetylating agent. Such a composition may also include at least one quaternary ammonium compound. Such a composition may generate peracetic acid (PAA) upon addition of water. The powdered composition, when dissolved at a concentration of 0.2% to 2% w/v in water can yield 200 to 2000 active ppm of peracetic acid that is stable for at least 24 hours, and can yield 400 to 4000 active ppm of hydrogen peroxide that is stable for at least 96 hours, and can also yield 80 to 1000 active ppm of quaternary ammonium chloride that is stable for at least 7 days.

In one embodiment, a powdered composition may include a peracetic acid precursor that includes about 30-80% w/w of a solid hydrogen peroxide precursor, about 10-40% w/w of a pH adjusting agent, and about 10-40% w/w of an acetylating agent. Such a powdered composition may contain 20 g of a solid hydrogen peroxide precursor, a pH adjusting agent, and an acetylating agent admixed with 1000 g of water to produce about 1000 to 3000 ppm of peracetic acid (PAA) generated in situ at pH 9.5±2.

In one embodiment, a powdered composition or formulation may include a peracetic acid precursor that includes about 30-80% w/w of a solid hydrogen peroxide precursor, about 10-40% w/w of a pH adjusting agent, and about 10-40% w/w of an acetylating agent, and may further include about 1-10% w/w of a powdered quaternary compound. Such a powdered composition may include about 0.01-10% w/w of a chelating agent. Such a powdered composition may contain 20 g of a solid hydrogen peroxide precursor, a pH adjusting agent, and an acetylating agent admixed with 1000 g of water to produce about 1000 to 3000 ppm of peracetic acid (PAA) generated in situ at pH 9.5±2, and 20 g of a powdered quaternary compound is admixed with 1000 g of water to produce about 800 to 1200 ppm of quaternary compounds released in water at pH 9.5±2.

In one embodiment, a powdered composition or formulation may include sodium percarbonate (technical, 85%) (CAS 15630-89-4), and citric acid (CAS 77-92-9), ethylenediamenetetraacetic acid (EDTA) EDTA-ACID (CAS 60-00-4), tetraacetylethylenediamine (TAED) (99.95%) (CAS 10543-57-4), Alkyl (60% C12, 30% C14, 5% C16, 5% C18) dimethyl benzyl ammonium chloride (CAS 68391-01-5), Alkyl (68% C12, 32% C14) dimethyl ethylbenzyl ammonium chloride (CAS 85409-23-0), and ethoxylated alcohol (NEODOL™91-8). The Alkyl dimethyl benzyl ammonium chloride and Alkyl dimethyl ethylbenzyl ammonium chloride are liquid and may then be dried on the surface of a urea prill.

Certain embodiments of a composition, including a powdered composition discussed herein and/or the solution resulting from such a composition, may be used to treat pathogens, such as bacteria, or pathogenic human and animal bacteria such as Clostridium difficile. Data and testing using certain embodiments of a composition, including a powdered composition discussed herein and/or the solution resulting from such a composition, may provide an effective treatment against HPV, ebola, anthrax, parvo, and norovirus.

In one embodiment, a method for the control of human and animal pathogens and/or the prevention of diseases associated with contamination of surfaces, wounds, tools, instruments and equipment may utilize an aqueous solution obtained by mixing water with a composition of water soluble ingredients comprising a peracetic acid precursor, such as hydrogen peroxide, hydrogen peroxide precursor or a mixture thereof, and a pH adjusting agent, and an acetylating agent. Such a composition may also include a chelating agent. Such a composition may also include at least one quaternary ammonium compound. Such a composition may generate peracetic acid (PAA) upon addition of water.

In one embodiment, a method for the control of animal pathogens where animals are housed in agricultural facilities, such as dairies, barns, farrowing pens, stalls, poultry houses, and the like, may utilize a composition or solution that may be comprised of water soluble ingredients comprising a peracetic acid precursor (such as hydrogen peroxide, hydrogen peroxide precursor or a mixture thereof), and a pH adjusting agent, and an acetylating agent. Such a composition or solution may also include at least one quaternary ammonium compound. Such a composition or solution may also include one or more surfactants. Such a composition may generate peracetic acid upon addition of water.

In one example, a stabilized formula containing peracetic acid and quaternary ammonium compounds was used to treat Clostridium difficile. Spores of Clostridium difficile were exposed for five (5) minutes to a 2.5% concentration containing precursors that generate peracetic acid in situ. Also spores of Clostridium difficile were exposed for five (5) minutes to a 2.5% concentration containing precursors that generate peracetic acid in situ and quaternary ammonium compounds. The efficiency of the composition containing peracetic acid, an acylating agent,and quaternary ammonium compounds are more effective than standalone actives. Thus, based at least in part on this experiment, there is a strong synergy between oxidizers (i.e., peracetic acid, hydrogen peroxide), acylating agent, and quaternary ammonium chloride in reducing the population of spores of C. difficile.

In one embodiment, it was discovered that when using a stabilized formula based on at least one oxidizer (e.g. peracetic acid, hydrogen peroxide) in combination with an acylating agent and at least one quaternary ammonium chloride (e.g. n-Alkyl (60% C14, 30% C16, 5% C12, 5% C18) Dimethyl Benzyl Ammonium Chloride and n-Alkyl (68% C12, 32% C14) Dimethyl Ethylbenzyl Ammonium Chloride), it is possible to obtain a synergistic activity between ingredients to provide a synergistic composition. Put another way, the addition of a quaternary ammonium salt to an oxidizer, like PAA or hydrogen peroxide in the presence of acylating agent, produces a better disinfectant than to be expected when adding the individual components.

This can enable a method and a use allowing to efficiently kill human and animal pathogens that infect surfaces, equipment and instruments, such as viruses, bacteria, yeast, fungus and mold. A stabilized composition can even completely eliminate MRSA, Anthrax and spores of Clostridium difficile at a concentration of 2 to 5% (w/v) with a contact time of 5 to 30 minutes. A stabilized composition may also be used to treat or eliminate E. Coli, Listeria, and Salmonella.

For example, this synergistic composition, method and use may be particularly efficient to kill human and animal pathogens such as viruses, bacteria, yeast and mold that may be present on the surfaces, equipment and instruments (e.g. endoscope) in medical and dental establishments, hospitals, veterinary buildings, and animal farms, as well as surfaces in dairy and food establishments.

Certain embodiments of the present invention may be used to produce a disinfectant fog. For example and not by way of limitation, an appropriate disinfecting solution may be used with an ultra low volume mechanical fogging apparatus to create small aerosol droplets.

As another example, an appropriate disinfecting solution may be used with an ultra low volume mechanical fogging apparatus to create small aerosol droplets and introduce an electrical charge to each droplet. This process may be referred to as electrostatic fogging. When the droplets are charged in such a process there is a two-fold effect. First, the droplets are attracted to surfaces. This allows the droplets to contact surfaces that are otherwise inaccessible to wipes, sprays, or other forms of fog. Second, when the droplets are charged a synergistic effect is observed between the liquid form of the compound in question and microbes. More specifically, the compound acts to kill microorganisms at a much higher rate if applied using an electrostatic fogger when compared to other ultra low volume foggers. A synergy is observed when the compound is formulated in a liquid solution then applied using an electrostatic fogger. When an electrical charge is applied to the liquid compound, the compound has a much greater antimicrobial activity against pathogens. Namely, the electrically charged particles act more quickly and more completely to kill pathogens.

It has been shown that certain compositions have little effect on tuberculosis. However, in certain embodiments, with the addition of a surfactant, there has been shown to be a 5 log reduction of tuberculosis. The surfactant helps facilitate the penetration of a waxy outer layer present on the tuberculosis microbe.

Certain embodiments of the present invention may be used to produce a disinfectant foam. Embodiments that include on or more surfactants in ratios sufficient to produce a thick foam may be used to produce a foam capable of standing on a solid surface. One purpose of such a foam may be to allow an even coating with the solution using minimal amounts of the solution. Such a preparation can be applied to hard surfaces and animals. Another purpose of such a foam may be to allow extended contact time in applications where the solution would otherwise dry upon the treatment surface, or run away from the treatment surface before the desired contact time was achieved.

In certain embodiments, a foam may be produced by using any suitable apparatus capable of producing a foam. For example and not by way of limitation, such an apparatus may provide air, which can be introduced using pneumatic tubing originating at a compressed air source and terminating at the apparatus. Such an apparatus may also include the input of a solution. Both solution and air may be introduced at a single point of entry.

Prior to the point of entry, a suitable solution may be prepared by the addition of a free flowing dry powder that may be added to a water container containing an agitator comprised of a circulating water pump, mechanical mixer, vortex, or fluid agitator. Within such a container the powder can be completely dissolved into a homogenous solution. Such a solution may then be pumped from the container using a water pump or pressure into the line that flows into the foaming apparatus.

At the foaming apparatus point of entry there may be a chamber where the air and solution are mixed and introduced into a foaming material comprised of foam, filter, mesh, sponge, lattice, or matrix. The pressure from the flow of solution and air may then result in the formation of foam generating from the end of the chamber.

Further, the apparatus used to produce and pump the solution may be automated wherein the solution is pumped from the container into the input lines only after a triggering event. The triggering event may result either from a timer or from a sensor which produces a signal which in turn opens a solenoid allowing the solution to flow from the pump into the foaming apparatus. A timer then allows the apparatus to produce foam for a period determined to produce an ideal amount of foam for the given application. Foaming times can vary depending on the treatment surface. Finally, foam may be generated using a ratio of air to solution as determined using gauge pressure at a ratio of solution 1, to air 2.

When preparing certain embodiments as described, a method for preparing a powder composition may be in the following order: first, add a dry peracetic acid precursor (hydrogen peroxide, hydrogen peroxide precursor, or a mixture thereof); second, an acetylating agent; third, a pH adjusting agent; fourth, a sequestering agent (or chelating agent); and, fifth, at least one quaternary ammonium compound. These chemicals may be mixed upon the addition of each chemical. Depending on the embodiment or intended use, not all chemical components may be needed or added. For example and not by way of limitation, a quaternary ammonium compound may not be added, and therefore not mixed into a composition. Further, the chemicals may be mixed in a shearing mixer or a vortex mixer in which the chemicals can be mixed to homogeneity without clumps.

When preparing certain embodiments as described, including an embodiment containing one or more surfactants, the method for preparing a powder composition may be in the following order: first, add a dry peracetic acid precursor (hydrogen peroxide, hydrogen peroxide precursor, or a mixture thereof); second, one or more surfactants, liquid or solid, with 99% or greater purity (anhydrous surfactants may be preferred so as to minimize reactivity during formulation and packaging); third, an acetylating agent; fourth, a pH adjusting agent; fifth, a sequestering agent (or chelating agent); and, sixth, at least one quaternary ammonium compound. These chemicals may be mixed first to the extent necessary to produce a completely homogenous mixture of peracetic acid precursor and surfactant. Then, once these two components are homogenous, the remaining chemicals may be each mixed upon its addition to the formula. Depending on the embodiment or intended use, not all chemical components may be needed or added. For example and not by way of limitation, a quaternary ammonium compound may not be added, and therefore not mixed into a composition. Further, the chemicals shall be mixed in a shearing mixer or a vortex mixer in which the chemicals can be mixed to homogeneity without clumps. Finally, it can be particularly important that the acetylating agent not be combined with the mixture until the liquids, namely one or more surfactants, are homogenously combined with the peracetic acid precursor. Moreover, when preparing certain embodiments as described, the surfactants can be pre-mixed to produce a homogenous solution prior to its addition to the powder peracetic acid precursor.

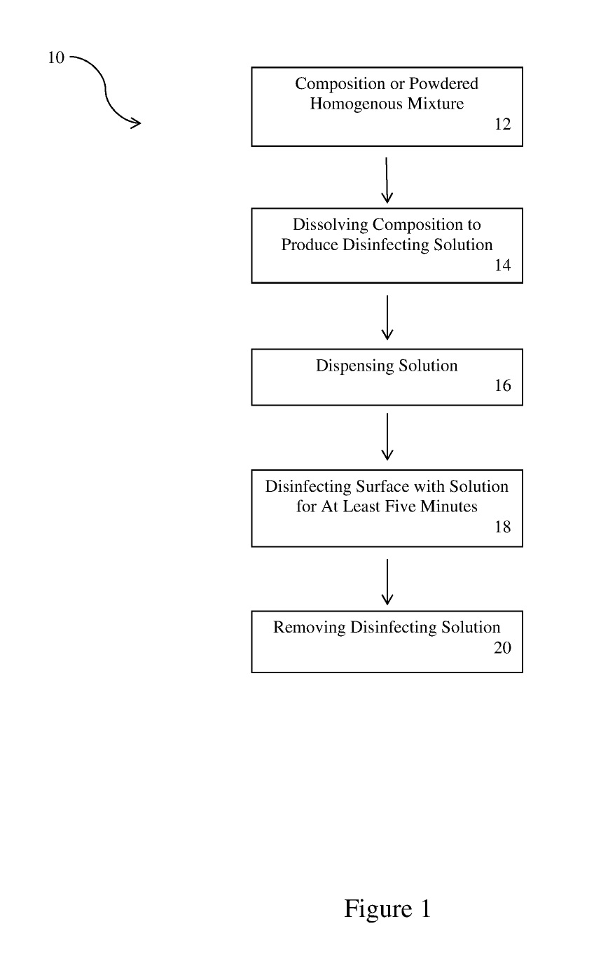

As shown in FIG. 1, a method 10 for disinfecting a variety of surfaces, tools, instruments or equipment may include providing or obtaining a composition or powdered homogenous mixture 12, dissolving the composition or powdered homogenous mixture to produce a suitable disinfecting solution 14, dispensing the disinfecting solution in an intended and suitable form 16, and disinfecting a surface with the disinfecting solution by allowing the disinfecting solution to remain on the surface for at least five (5) minutes, or as required to disinfect the surface or comply with appropriate regulations 18. In certain embodiments, the disinfecting solution will be removed from the surface 20, but in other embodiments, the disinfecting solution does not need to be removed.

When providing or obtaining a composition or powdered homogenous mixture 12, any suitable embodiment, and/or process for preparing such an embodiment, described herein may be utilized. For example and not by way of limitation, a composition or powdered homogenous mixture 12 may include an appropriate amount of sodium percarbonate, and an appropriate amount of citric acid, and an appropriate amount of ethylenediamenetetraacetic acid (EDTA), and an appropriate amount of tetraacetylethylenediamine (TAED) (99.95%), and an appropriate amount of alkyl dimethyl benzyl ammonium chloride, and an appropriate amount of alkyl dimethyl ethylbenzyl ammonium chloride, and an appropriate amount of ethoxylated alcohol. Such a composition may be prepared in a manner that results in a powdered, homogenous mixture as described herein.

When dissolving the composition or powdered homogenous mixture to produce a suitable disinfecting solution 14, generally, the composition will be dissolved in an appropriate amount of water. Various and suitable blending machines may be used to help make sure the composition is dissolved appropriately.

When dispensing the disinfecting solution in an intended and suitable form 16, the disinfecting solution may be dispensed in a variety of ways depending on the intended use and environment. Such dispensing may include without limitation, disinfecting solution dispensed as a spray, a fog, and/or a foam. Disinfecting solution may be dispensed as a spray utilizing any suitable spraying means, such as a spray bottle, sprinkler, spray nozzle, or the like. Disinfecting solution may be dispensed as a fog utilizing any suitable fogging means, such as an ultra low volume mechanical fogging apparatus, or an ultra low volume mechanical fogging apparatus that introduces an electrical charge to each droplet (electrostatic fogger) as described herein. Disinfecting solution may be dispensed as a foam utilizing any suitable foaming means, such as any suitable apparatus where the air and solution are mixed so as to introduce a foaming material as described herein, or any similar apparatus.

When disinfecting a surface with the disinfecting solution by allowing the disinfecting solution to remain on the surface for at least five (5) minutes, or as required to disinfect the surface or comply with appropriate regulations 18, the disinfecting solution is allowed to remain on the surface, tool, instrument, or equipment for any amount of time required to disinfect the surface. Such a time may be dictated by appropriate statute or may be the time necessary to complete disinfection as desired.

Moreover, the disinfecting solution may be removed from the surface 20, but the disinfecting solution does not need to be removed in every instance. For example, disinfecting solution dispensed as an electrostatic fog may not need to be removed after disinfection. In other embodiments, the disinfecting solution may be removed by rinsing the surface, tool, instrument or equipment with potable water. In other embodiments, the disinfecting solution may be removed by wiping the surface clean.

Certain embodiments of the present invention may be used for disinfecting surfaces and equipment in agricultural buildings. For terminal cleaning and disinfection in agricultural buildings, all surfaces are saturated with a minimum 0.2% to 2% w/v solution of the stabilized composition (2 g to 20 g per 1L). This concentration yields 200 to 2000 active ppm of peracetic acid stable for at least 24 hours, and it also yields 400 to 4000 active ppm of hydrogen peroxide stable for at least 96 hours, and it also yields 80 to 1000 active ppm of quaternary ammonium chloride stable for at least 7 days. The stabilized composition may remain in contact with the surface to be disinfected for at least 10 minutes.

Soiled utensils may be scrubbed heavily and soaked in a 0.2% to 2% w/v solution of the stabilized composition for 10 minutes, and should not exceed 45 minutes for metal objects. A concentration of 2 g to 20 g per 1L yields 200 to 2000 active ppm of peracetic acid stable for at least 24 hours, and it also yields 400 to 4000 active ppm of hydrogen peroxide stable for at least 96 hours, and it also yields 80 to 1000 active ppm of quaternary ammonium chloride stable for at least 7 days.

Certain embodiments of the present invention may be used for disinfecting surfaces and equipment in veterinary hospitals. For example, surfaces to be disinfected may be thoroughly wet with a 0.2% to 2% w/v solution of the stabilized composition. A concentration of 0.2% to 2% w/v solution of the stabilized composition yields 200 to 2000 active ppm of peracetic acid stable for at least 24 hours, and it also yields 400 to 4000 active ppm of hydrogen peroxide stable for at least 96 hours, and it also yields 80 to 1000 active ppm of quaternary ammonium chloride stable for at least 7 days. A minimum contact time of 10 minutes is recommended, or as required.

Certain embodiments of the present invention may be used for disinfecting laboratory equipment and bowls. For example, soiled utensils may be scrubbed heavily and soaked in a 0.2% to 2% w/v solution of the stabilized composition for a minimum of 10 minutes, and one should not exceed 45 minutes for metal objects).

Certain embodiments of the present invention may be used for disinfecting surfaces and equipment in food processing plants. The stabilized composition may be used on previously cleaned equipment. The stabilized composition may be used at a concentration of 0.2% to 2% w/v solution of the stabilized composition, for example, 2 g to 20 g per 1L. This solution is kept in contact with food processing surfaces for at least 10 minutes, then rinsed abundantly with potable water. If peracetic acid concentration is equal to or below 200 ppm, it need not rinsed.

Certain embodiments of the present invention may be used for disinfecting surfaces and equipment in dairies, including the treatment of animals, and for the treatment of biofilm, bacteria, fungi, and other pathogens in milk lines, storage tanks, chillers, and other hardware whether in contact with milk or water.

Certain embodiments of the present invention may be used for disinfecting surfaces and equipment in medical and dental establishments. The stabilized composition may be used on previously cleaned equipment and medical instruments. The stabilized composition may be used at a concentration of 1.0% to 3% w/v solution of the stabilized composition, for example, 10 g to 30 g per 1L. This solution is kept in contact with surfaces and equipment for at least 10 minutes, then rinsed abundantly with potable water.

Certain embodiments of the present invention may be used for animal treatment. Currently animals are being treated with products such as copper sulfate, chlorine, formalin (formaldehyde), and chlorhexidine gluconate to treat and prevent infection. These treatments have proven dangerous to the environment and pose a significant health risk to users.

Certain embodiments of the present invention may replace these chemicals for the treatment of bacterial, viral, fungal, and similar infections in animals. Treatment of such animals may include exposure to a 0.4% - 2.0% w/v solution, containing a pH balancer and one or more surfactants. This treatment may be in the form of a liquid bath, spray, or foam. Studies have shown a significant improvement over competing products, such as Copper Sulfate and Formalin. In comparative studies, animal health showed a significant improvement over copper sulfate when animal populations were treated with this composition.

Certain embodiments of the present invention may be used for aerial disinfection. For example, to replace dangerous and ineffective formalin fumigation in empty farm buildings and veterinary hospitals. The ventilation system is shut down during the disinfection. A mechanical fogging machine may be used as part of a terminal disinfection routine with a 0.2 to 1.2% w/v solution of the stabilized composition. It may be applied at a rate of one liter of solution per 100 m3 with particle size not exceeding 70 microns in order to get a minimal contact time of 10 minutes with microorganisms in the air. The room or area should be unoccupied during the fogging. Users and animals may re-enter the treated area once the fog has dispersed. Usually, no rinsing is required after fogging.

Numerous tests have been performed evaluating the disinfecting capabilities of various embodiments of the present invention and various concentrations of disinfecting solutions obtained utilizing the descriptions contained herein. The following examples are provided as illustrative and not by way of limitation.

In ten (10) minutes, a 5% disinfecting solution will kill more than 99.9999% of Clostridium difficile spores on a hard surface. A standard for disinfection testing, Clostridium difficile is known to produce some of the most hard to kill bacterial spores of all bacteria. Tests were performed using AOAC testing methods, MB-05-14, MB-28-04, MB-31-03.

In three (3) minutes, a 1% disinfecting solution will kill more than 99.9999% of Staphylococcus aureus (MRSA) on a hard surface. In three (3) minutes, a 1% disinfecting solution will kill more than 99.9999% of Vancomycin-Resistant Enterococcus (VRE) on a hard surface. In three (3) minutes, a 1% disinfecting solution will kill more than 99.9999% of Pseudomonas aeruginosa on a hard surface. Tests were performed using AOAC testing method MB-05-14.

In fifteen (15) seconds, a 5% disinfecting solution will kill 99.9999% of Staphylococcus aureus. In fifteen (15) seconds, a 5% disinfecting solution will kill 99.9999% of Escherichia coli on a hard surface. Tests were performed using AOAC testing method MB-05-14.

In three (3) minutes, a 5% foaming formula will kill 98.6%, and in five (5) minutes 99.986%, of Mycobacterium bovis (Tuberculosis) on a hard surface. Mycobacterium bovis is known as one of the most difficult bacteria to kill. Tests were performed using AOAC testing method MB-16-02.

When tested against Copper Sulfate (5% solution), a 5% disinfecting solution killed about twice as many spirochaete bacteria. Spirochaete bacteria were isolated and observed using bioluminescent technology and dark field microscopy, a technique that is unique to spirochaete, because the bacteria cannot be assayed in a common petri dish.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A composition use in disinfecting applications comprising:
an oxidizer precursor in powdered form;
a pH adjusting agent in powdered form; and
an acetylating agent in powdered form, wherein the oxidizer precursor, the pH adjusting agent, and the acetylating agent are mixed together to produce an homogenous mixture.

2. The composition of claim 1, further comprising a chelating agent in powdered form, wherein the chelating agent is mixed into the homogenous mixture.

3. The composition of claim 2, further comprising a quaternary ammonium salt in powdered form, wherein the quaternary ammonium salt is mixed into the homogenous mixture.

4. The composition of claim 2, wherein the homogenous mixture is mixed with water to produce an oxidizing, disinfecting solution.

5. The composition of claim 1, wherein the oxidizer precursor comprises about 30-80% w/w of the homogenous mixture, the pH adjusting agent comprises about 10-40% w/w of the homogenous mixture, and the acetylating agent comprises about 10-40% w/w of the homogenous mixture.

6. The composition of claim 5, further comprising a chelating agent that comprises about 0.01-10% w/w of the homogenous mixture.

7. The composition of claim 6, further comprising a quaternary ammonium salt that comprises about 1-10% of the homogenous mixture.

8. The composition of claim 1, wherein the oxidizer precursor is selected from the group consisting of a peracetic acid precursor, a hydrogen peroxide precursor, a persalt, or hydrogen peroxide.

9. The composition of claim 3, wherein the quaternary ammonium salt is selected from the group consisting of benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, didecyl dimethyl ammonium bromide, or dioctyl dimethyl ammonium bromide.

10. The composition of claim 2, further comprising a surfactant, wherein the surfactant is included in the homogenous mixture.

11. A composition used in disinfecting applications comprising:
sodium percarbonate;
citric acid;
ethylenediamenetetraacetic acid;
tetraacetylethylenediamine;
alkyl dimethyl benzyl ammonium chloride;
alkyl dimethyl ethylbenzyl ammonium chloride; and
ethoxylated alcohol, wherein the sodium percarbonate, citric acid, ethylenediamenetetraacetic acid, tetraacetylethylenediamine, alkyl dimethyl benzyl ammonium chloride, alkyl dimethyl ethylbenzyl ammonium chloride, and ethoxylated alcohol are mixed together to form a homogenous mixture in the form of a powder.

12. The composition of claim 11, further including water to form a solution.

13. The composition of claim 11, wherein the homogenous mixture further includes a surfactant.

14. The composition of claim 13, further including water to form a solution.

15. A method for disinfecting surfaces, instruments, or equipment comprising:
providing a homogenous mixture, wherein the homogenous mixture comprises an oxidizer precursor in powdered form, a pH adjusting agent in powdered form, and an acetylating agent in powdered form;
adding water to the homogenous mixture to produce a disinfecting solution;
dispensing the disinfecting solution; and
allowing the disinfecting solution to remain on a treatment surface as dispensed for at least five minutes.

16. The method of claim 15, wherein the homogenous mixture further includes a chelating agent in powdered form and at least one quaternary ammonium salt in powdered form.

17. The method of claim 16, wherein the homogenous mixture further includes a surfactant.

18. The method of claim 16, wherein the disinfecting solution is dispensed as a fog.

19. The method of claim 18, wherein the disinfecting solution is dispensed as a fog using an electrostatic fogging machine.

20. The method of claim 17, wherein the disinfecting solution is dispensed as a foam.
